(19) United States
(12) Patent Application Publication
(10) Pub. No.: US 2008/0310584 A1
(43) Pub. Date: Dec. 18, 2008

(54) Tomography Equipment Comprising a Variable Reproduction Geometry

(75) Inventors: Joachim Hey, Bornheim (DE); Dirk Freyer, Bonn (DE); Manfred Breuer, Alfter (DE); Gerhard Zundorf, Bonn (DE); Marc Lievin, Bonn (DE)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: SIRONA DENTAL SYSTEMS GMBH

(21) Appl. No.: 11/883,418

(22) PCT Filed: Jan. 31, 2006
(86) PCT No.: PCT/EP06/00842

§ 371 Date: Jul. 31, 2007

(30) Foreign Application Priority Data
Jan. 31, 2005 (DE) 10 2005 004 502.2

(51) Int. Cl. A61B 6/00

(52) U.S. Cl. 378/15

(57) ABSTRACT

The invention relates to a method for generating 3D tomographic images of an object (4), according to which a radiation source (1), in particular an X-ray source is displaced in relation to the object (4). The radiation source (1) emits a conical beam of radiation (2) that strikes the object (4), the radiation that has passed through the object (1) and has been weakened in intensity is captured by a detector (5); which is located in, he conical beam (2) behind the object (4) in relation to the radiation source (1). The radiation source (1) and the detector (5) are combined in a source-detector assembly (7), which is rotated about a rotational axis (6) during the generation of images in the reference system that is defined by the object (4). Said rotational axis (6) is modified during the generation of the images and/or the source (1) and/or the detector (5) in the reference system that is defined by the source-detector assembly (7) is/are displaced during the generation of the images.

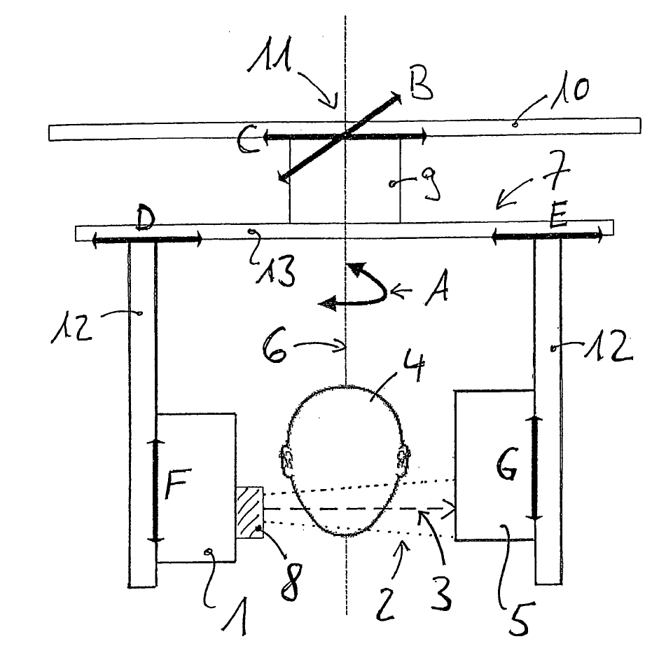

The present invention relates to a method of preparing a sequence of individual images from which 3-D tomographic images of an object are produced, whereby a source of radiation, more particularly an X-ray source is displaced in relation to the object, whereby the source of radiation emits radiation in a conical beam of radiation which strikes the object, whereby the radiation passing through the object and weakened in intensity is recorded by a detector which is arranged in relation to the radiation source in the conical beam of radiation behind the object, whereby the source of radiation and the detector are combined in a source-detector assembly which during the generation of a sequence is rotated about a axis of rotation in the reference system defined by the object. The invention also relates to a device for implementing such a method.

Particularly in the field of medicine, tomographic images are an important method of producing three-dimensional images of parts of the human body. In order to do this the part of the body is exposed to a source of radiation, more particularly a source of X-rays, which is moved in a pre-determinable path, usually a circle or an ellipse, around the object. In the conical beam of radiation of the source detector comprising an array of detector elements and onto which the object is projected is arranged behind the object.

In general these methods, which are used in computer tomography and in cone-beam tomography, allow the reconstruction of the three-dimensional distribution of the absorption coefficients of the object penetrated by the radiation from the absorption measurements or from the individual images. With regard to this the use of this method is not only restricted to the field of medicine.

In the known conical beam tomography devices the source of radiation and the detector move on elliptical paths or on circular paths about a common isocentre in which the object to be reproduced is situated. In a preferred embodiment the X-ray source and the detector are each arranged on the end of a C-arc which is rotated about its centre axis around the object. A number of two-dimensional projections are recorded from which the object is reconstructed in its three-dimensional state. In order to reconstruct three-dimensional objects the primary beam technique by Feldkamp (“Practical Primary Beam Algorithms” by F. A. Feldkamp et al. J. Opt. Soc. Am. A/edition 1, no. 6, June 1984) can be used. In this method, which is also known by the term “filtered back-projection” (FBP) all projection images are initially filtered and them projected back in their spatial form. The method is used in commercial tomographic scanners, in particular spiral CTs or primary beam C-arms.

Such a diagnosis device is set out, for example, in EP 1 000 582 A2 whereby this C-arc has the special feature that its source-detector assembly is designed to compensate for movements of the device, such as vibrations, in order to guarantee constant precise orientation of the axis of rotation (isocentric rotation). The movements of the device are determined -via sensor, the output signals of which control corresponding actuators.

A disadvantage of the methods known up to now is that they are relatively inflexible in terms of their arrangement and can hardly adapt to the spatial conditions defined by the object to be examined. They are designed for various applications including conical beam tomographs with different reproduction geometries in order to optimise the costs and spatial requirements of the devices. In particular, with the known methods it is difficult to take into account the individual anatomy of the patient. Thus, in many respects compromises are necessary as a result of which the reproduction quality, the radiation exposure and thereby the patient have to suffer.

The aim of the invention is to create a generic method that can be implemented with simple means and guarantees great flexibility with regard to the geometry of the objects to be examined at low radiation exposure. It is also the aim of the invention to create a mechanically simply designed device to implement the method.

These tasks are solved by the method with the characterising features of claim 1 and the device in accordance with claim 8.

Features of particular forms of embodiment of the invention are set out in the respective dependent claims.

The essential underlying concept of the invention consists in using as part of the method as many degrees of freedom of movement as possible of the source and detector in relation to the object to be reproduced in a controlled manner during imaging without making too great demands on the performance of the evaluation programs with the complexity of movement. At the same time the invention consists in implementing this basic concept in a structurally simple and in practice reliably operating device. This possibility is opened up in that during a sequence the axis of rotation itself, about which the source-detector assembly rotates, is changed actively and, particularly, in relation to the object to be reproduced, whereby this change consists in a parallel displacement and/or pivoting of the axis of rotation. Movement of the source-detector assembly along the axis of rotation as is known for the generation of a spiral, does not constitute this “change” in accordance with the invention as the axis of rotation remains fixed.

In addition or alternatively thereto, during the generation of the sequence, i.e. during recording, the source and/or the detector are moved towards each other within the source-detector assembly designed as a structural unit. The rotatably arranged source-detector assembly thus forms a rotating reference system in which the source and/or detector are moved. This movement can be a pivoting movement or constitute a displacement in parallel and/or radially to the axis of rotation of the source-detector assembly. The source can also be arranged differently with regard to the detector during the scan.

Expressed in another way, in accordance with the invention—in contrast to conventional conical beam tomography devices—the axis of rotation can be displaced and/or pivoted with regard to the object to be examined during an imaging sequence. Additionally or alternatively thereto the detector and source of radiation can be moved radially towards or away from the current axis of rotation. Here it is also advantageous if the detector and source of radiation can also be moved in parallel to the current axis of rotation during imaging. In principle the invention permits any type of path (scanning curves) on which the source of radiation and the detector move. It must of course be ensured that the source and detector are at all times oriented towards each other that the radiation strikes the active surface of the detector. The computer functionality required to evaluate the image produced along the scanning curve must be provided.

The advantages of the method in accordance with the invention and the corresponding device, which offers a greater degree of freedom with regard to the relative position of the source of radiation, object and detector are evident: the advantages compared with a conventional conical beam tomography device are the better resolution and better contrast within the reconstructed volume, the lower radiation exposure of the object, more particularly the patient, the enlargement of the reconstructable volume and the reduced spatial requirement of the tomography device. With the invention the imaging geometry for each individual image can be optimised with regard to the following factors: thus, depending on the geometry of the object to be reproduced settings for a sequence can be selected in which the movement of the source of radiation and detector are not disruptive. The movement can thus be adapted to the contours of the object, more particularly the body part. Due to the relatively great flexibility with regard to the dimensions of the object, devices with a more compact mechanical structure can be designed, which is reflected positively in the costs and spatial requirement of the tomography device.

With the invention it can also be guaranteed that the relevant parts of the object, for which a 3D distribution is to generated, are recorded as completely as possible by the conical beam of radiation in each image. On the other hand the movement can be set so that the non-relevant parts of the object, for which no 3D distribution is to be generated, are not as far as possible recorded by the conical beam of radiation. In this way unnecessary dose exposure and absorption are avoided whereby the lower dose exposure is beneficial to the patient and the reduced absorption results in significantly less image interference and thus a higher image quality. In this way anatomical structures with a high coefficient of absorption can be investigated without problems.

A further advantage of the invention is that the relevant anatomical structures can be homogeneously irradiated in the sense that the integrated absorption along all individual beams from the source of radiation through the detector to the object are approximately of equal size. In this way maximum dynamics and thereby contrast are assured at minimal dose. In addition, through the geometrical arrangement, i.e. through the relationship of the distance between the detector and source of radiation and the distance between the source of radiation and the object, the enlargement and thereby the resolution of the 2D individual images and thereby in turn the resolution can be influenced in the reconstructed 3D volume.

The resolution of the individual images also depends on the geometrical extent of the source of radiation. Thus, the extent of the source or radiation in the case of X-ray sources is the X-ray focus on the anode. The influence of the spatial extent of the source increases with increasing enlargement and the resolution decreases. The advantage of the invention now lies in the fact that in accordance with the requirements for each conical beam tomography device the optimum reproduction geometry can be found, as the movement is not limited to a circle and ellipse.

It is also advantageous if the movement of the individual components, as well as the positions at which individual images are recorded, and the aperture settings are defined before a sequence of images, and set during the sequence in computer-controlled and motor-driven manner. In order to minimise the mechanical stresses on the device associated with acceleration of the components, it is advantageous to continuously carry out movements brought about by the motors, in particular the step motors, during recording. However, for certain applications it may be advantageous to carry out the movement of source and detector in steps, whereby the step-width and rest time after a step can be adjusted. If necessary individual images from a sequence can be produced with different doses of radiation, which can lead to a higher resolution with regard to the densities and an improved contrast within the object to be examined. With the known advantages a preferred area of application for the device in accordance with the invention is dental and maxillary diagnosis.

Advantageously the images produced with the device in accordance with the invention are planned beforehand on the computer by way of simulations. In this way with conventional methods recorded volume data from the patient can form the basis of the simulation. The results of the simulation are then used to control the actuators of the device in accordance with the invention.

It is also possible to carry out patient-specific sequences which take into account various individual anatomies such as corpulent, thin, tall or small. These sequences can be planned on the basis of the external anatomy of the patient which has been previously recorded (optically or with the device mechanics). It is also possible to travels the paths beforehand manually and save them directly for a subsequent scan.

In order to illuminate the detector optimally with the emitted conical beam of radiation without exposing unnecessary parts of the object it is also advantageous to provide the source of radiation with an adaptive aperture device, the opening geometry of which can be adjusted in a motor-driven and computer-controlled manner.

In accordance with the invention it is advantageous if the position of the individual images is selected so that one of the known reconstruction methods, in particular the above primary beam method for reconstruction of the 3D distribution can be used. For this the geometry has to be calibrated for each individual image in order to be able to reconstruct the three dimensional image correctly. Calibration can take online during the scan or offline, whereby offline calibration is carried out once using a reference object, as described, for example in U.S. Pat. No. 6,715,918.

As has already been indicated, a third advantage consists in the fact that with the increased flexibility in movement and the associated improved adaptation to anatomical circumstances, certain anatomical structures such as, for example, the base of the skull, which are particularly sensitive or strongly absorb radiation, can be kept out of the beam of radiation. In this way measuring artefacts formed by these anatomical structures are avoided. In this way the radiation dose for the patient can be reduced while retaining the same image quality.

It is also advantageous if he artefacts produced by metal objects can be reduced. Such metal artefacts caused by increased absorption (occlusions), as brought about in particular by dental fillings can be avoided with C-arc. Thus, strongly absorbing objects can completely absorb the X-ray radiation which in the recorded data set is seen as a lack of information. This lack of information then causes artefacts particularly if the classical algorithms for reconstruction are used in which the process of back projection consists of a summation. In the case of occlusions the summation is inconsistent and the values become saturated outside the permissible range. In summary, the essential concept of the invention lies in the special arrangement which during imaging allows the source, the detector, the axis of rotation and/or the isocentre to consciously move relative to each other in all degrees of freedom. With any arrangement of the scanning curve optimum adaptation to the requirements, in particular the patient anatomy, homogeneous irradiation, the radiation exposure, the image quality, the reconstructed volume and the available space is possible. A rotation isocentre is no longer a prerequisite with the invention.

A form of embodiment of the invention will be explained in more detail below with the aid of the figure.

The figure schematically shows a primary beam scanning system with a source 1 of X-ray radiation. The radiation emerges from the source 1 in a conical beam of radiation 2 with a central beam 3. In this case the source of radiation 1 is provided with a motor-driven and computer-controlled adaptive aperture 8 which limits the conical beam of radiation 2. With the conical beam of radiation 2 an object 4, in this case the head of a standing patient, is irradiated whereby the beam, which is weakened in intensity as it passes through the head 4, strikes a detector 5 which on its active surface has a number of individual detector elements. Each of these detector elements takes up a weakened partial beam of the conical beam of radiation 2. To generate a three-dimensional reconstruction of the head 4 the assembly comprising the source 1 and detector 5 is rotated about the axis 6 (arrow A) whereby the source 1 and the detector 5 are combined in a rotating source-detector assembly 7 similar to a C-arc. While moving the detector generate individual images of the radiation weakened by passing through the object. The detector can be an X-ray image intensifier or a flat panel detector.

With the adaptive aperture 8 which can be designed as a multi-leaf collimator, it is possible to continuously adjust the direction and extent of the conical beam during an imaging sequence. Advantageously the angle of the central beam 3 is set during imaging so that it strikes the middle of the detector 4.

In accordance with the invention in the shown device there are several degrees of freedom for the relative movement of the components source 1 and detector 5 in the references system of the head. Initially the complete source-detector assembly 7, which is attached via a holder 9 on a cover plate 10 or a frame, can be moved forward in a motor-driven manner in the coordinate system 11, mounted from axes B and C, in the plane of the cover plate 10. The cover plate 10 does not necessarily have to be vertically orientated. Its slope can also be adjusted. Displacement in the plane of the cover plate 10 takes place during the imaging of a sequence, i.e. in this case during rotation about the axis of rotation 6. The displacement, in this case a parallel displacement of the rotation axis, is planned and set in advance of the imaging sequence. A computer then takes over the control of the predetermined movement during imaging.

In this case degrees of freedom are also provided in the reference system of the source-detector assembly 7. Thus, the source 1 and the detector 5 are each attached to a holder arm 12, which is suspended in a displaceable manner in a holder 13, whereby the joint holder 13 is perpendicularly orientated with regard to the axis of rotation 6. In this way independent displacement of the source 1 and the detector 5 is possible in the plane of the holder 13 along arrows D and E.

Additional degrees of freedom are achieved in that both the source 1 and the detector 5 can be moved along the holder (arrows F and G).

It is also conceivable to arrange source 1 and detector 5 in a tilting manner. Through tilting the gradient of source 8 and the detector 5 can be selected so that the lower edge of the beam is horizontal. With this arrangement irradiation of the shoulder is avoided and the detector 5 can be moved relatively close to the patient.

The movements of source 1 and detector 5 along arrows D, E, F and G or tilting are planned and set in advance of the imaging, whereby a computer again carries out the control of the predetermined movements during imaging.

1.-9. (canceled)

10. Method of generating 3-D tomographic images of an object (4), whereby a source of radiation (1), more particularly an X-ray source is displaced in relation to the object (4), whereby the source of radiation (1) emits radiation in a conical beam of radiation (2) which strikes the object (4), whereby the radiation passing through the object (1) and weakened in intensity is recorded by a detector (5) which is arranged in relation to the radiation source (1) in the conical beam of radiation (2) behind the object (4), whereby the source of radiation (1) and the detector (5) form a source-detector assembly (7) which during the generation of images is rotated about a axis of rotation (6) in the reference system defined by the object (4), whereby the axis of rotation (6) is changed during the generation of images and/or that the source (1) and/or the detector (5) is/are moved in the reference system defined by the source-detector assembly (7) during the generation of the images, and wherein the source of radiation (1) and/or the detector is/are continuously moved perpendicularly to the axis of rotation and relative to the object (4) during an imaging sequence.

11. Method in accordance with claim 10, wherein the conical beam of radiation (2) is continuously adjusted in terms of its direction and extent by an adaptive aperture (8) during an imaging sequence.

12. Method in accordance with claim 11, wherein the angle of the central beam (3) is adjusted during imaging in order to strike the centre of the detector (5).

13. Device comprising a source of radiation (1) and a detector (4), whereby the source of radiation (1) and the detector (4) are combined into a source-detector assembly (7) whereby the source- detector assembly (7) is arranged in rotating manner about an axis of rotation (6), wherein the source-detector assembly (7) is borne in a displaceable manner perpendicularly to the axis of rotation (6), whereby drive means for the source-detector assembly (7) are provided that bring about a displacement during the imaging of a sequence, whereby the drive means are controlled by a computer.

14. Device in accordance with claim 13, wherein the detector (5) is an X-ray image intensifier or a flat panel detector.

15. Device in accordance with claim 13, wherein the source (1) and/or the detector (5) is/are held in a movable manner on the source-detector assembly (7), whereby drive means are provided for the source (1) and/or the detector (5) which bring about a displacement during the imaging of a sequence.
